(19) United States
(12) Patent Application Publication
(10) Pub. No.: US 2011/0313394 A1
(43) Pub. Date: Dec. 22, 2011

(54) Sensor Controlled Flow Path For Providing Fluids To Patients

(75) Inventor: Donald E. Bobo, SR., Fountain Valley, CA (US)

(73) Assignee: INNERSPACE, INC, Tustin, CA (US)

(21) Appl. No.: 13/129,574

(22) PCT Filed: Oct. 26, 2009
(86) PCT No.: PCT/US09/62138

§ 371 Date: Sep. 12, 2011

Related U.S. Application Data
(60) Provisional application No. 61/197,040, filed on Oct. 24, 2008.

(51) Int. Cl. A61M 5/168

(52) U.S. Cl. 604/504; 604/151; 604/66; 604/500

(57) ABSTRACT

An outflow line is connected to an infusion pump that is programmed to run at a fixed rate. The outflow line has a bypass line that leads from the pump outlet back to the pump inlet. A line downstream of the entrance to the bypass line leads to the patient. The system has two valves. The fluid en route to the patient passes through the first valve. The bypass line passes through the second valve. When one valve is open, the other is closed. The valves' flow states are controlled by a signal from a control instrument. The system allows the pump output to be directed to the patient or to a bypass circuit that returns fluid leaving the pump back to the pump inlet. The system makes it possible to vary the amount of fluid delivered to a patient by an infusion pump set at a constant pumping rate by varying the open and closed state of valves in response to a signal from a patient monitoring instrument.

### RELATED APPLICATIONS

This application claims priority to U.S. Provisional Application Ser. No. 61/197,040 filed Oct. 24, 2008, entitled Sensor Controlled Flow Path of IV Pump Fluid, which is hereby incorporated herein by reference.

### FIELD OF THE INVENTION

The present invention relates to systems and methods for providing fluids to patients, and more particularly to sensor controlled fluid flow paths and methods of use thereof.

### BACKGROUND OF THE INVENTION

Infusion pumps are commonly used to provide liquid medications and/or nutrients to patients. While infusion pumps are typically used for intravenous (IV) therapy, therapies in which liquid substances are infused directly into a vein, infusion pumps may also be used for subcutaneous, arterial and epidural infusions. There are numerous situations in which medical treatments benefit from or may be optimize by linking the administration of a medication into a patient to a patient sensor output.

For example, high and low blood glucose levels can place certain patients at risk. Blood glucose levels are reduced to an acceptable level by administering insulin via IV therapy. Close control of blood glucose requires that the insulin flow rate be increased or decreased to regulate the amount of insulin infused into the patient. In a hospital setting, a nurse must frequently monitor blood glucose levels and adjust the infusion pump rate up or down accordingly. This manual approach, in one extreme, is very time consuming, and in another extreme, places the patient at risk if the glucose level is not closely monitored and the flow rate properly adjusted. An obvious automated or semi-automated approach to handling this situation is to control insulin delivery to the patient by automatically adjusting the fluid output of the infusion pump by changing the pumping speed in response to a signal from a control unit that receives input from a glucose sensor. The concept of varying the infusion pump speed is, however, impractical for several reasons. First, the matter of replacing the population of infusion pumps in service with pumps that change output in response to information from a monitoring instrument would be prohibitively expensive. Second, the logistics of having a limited number of specialty pumps available where needed would be complicated as pumps are typically moved freely throughout a hospital.

Hence, there is a need for a fluid delivery system that can change the rate at which IV therapy fluid is delivered to a patient by an infusion pump that runs at a constant pump speed when a change in flow rate is indicated by a sensor of an instrument that analyzes flow requirements.

### OBJECTS AND SUMMARY OF THE INVENTION

The present invention provides a fluid delivery system that can change the rate at which IV therapy fluid is delivered to a patient by an infusion pump that runs at a constant pump speed when a change in flow rate is indicated by a sensor of a control instrument that analyzes flow requirements.

### BRIEF DESCRIPTION OF THE DRAWINGS

These and other aspects, features and advantages of which embodiments of the invention are capable of will be apparent and elucidated from the following description of embodiments of the present invention, reference being made to the accompanying drawings, in which:

FIG. 1 shows system components and a fluid flow path of certain embodiments of the present invention when it is intended that a fluid be provided to a patient.

FIG. 2 shows system components and a fluid flow path of certain embodiments of the present invention when it is intended that a fluid flow to a patient be stopped.

FIG. 3 shows system components and a fluid flow path of certain embodiments of the present invention when it is intended that fluid flow through a patient line be stopped and only a reduced fluid flow be maintained to the patient.

### DESCRIPTION OF EMBODIMENTS

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention. In the drawings, like numbers refer to like elements.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

The present invention provides solutions for controlling the amount of fluid delivered to a patient by an infusion pump that runs at a fixed rate. As a general overview, the fluid passing through the pump is either delivered to the patient or is directed back to the inlet of the infusion pump. The flow path of the fluid is determined by the flow state of two valves. One valve controls fluid flow through a patient line and the other valve controls fluid flow through a bypass line. If one valve is open, the other is closed. The valves' flow states are controlled by a signal sent from a control instrument that, in conjunction with a sensor, monitors a patient parameter, such as blood glucose. The present invention makes it possible to vary the amount of fluid delivered to a patient by an infusion pump set at a constant pumping rate by varying the valves' open and closed states in response to a signal from the control instrument.

In some applications, it is necessary to maintain a continuous, low level of fluid flow through the patient line to prevent the needle or catheter from clotting closed. Accordingly, in certain embodiments of the present invention, a keep the vein open (“KVO”) flow requirement is achieved by employing a KVO bypass line that bypasses the valve in the patient line. A flow resistor is employed within the KVO bypass line that allows a limited quantity of fluid to pass through the patient line regardless of the state of the patient line valve.

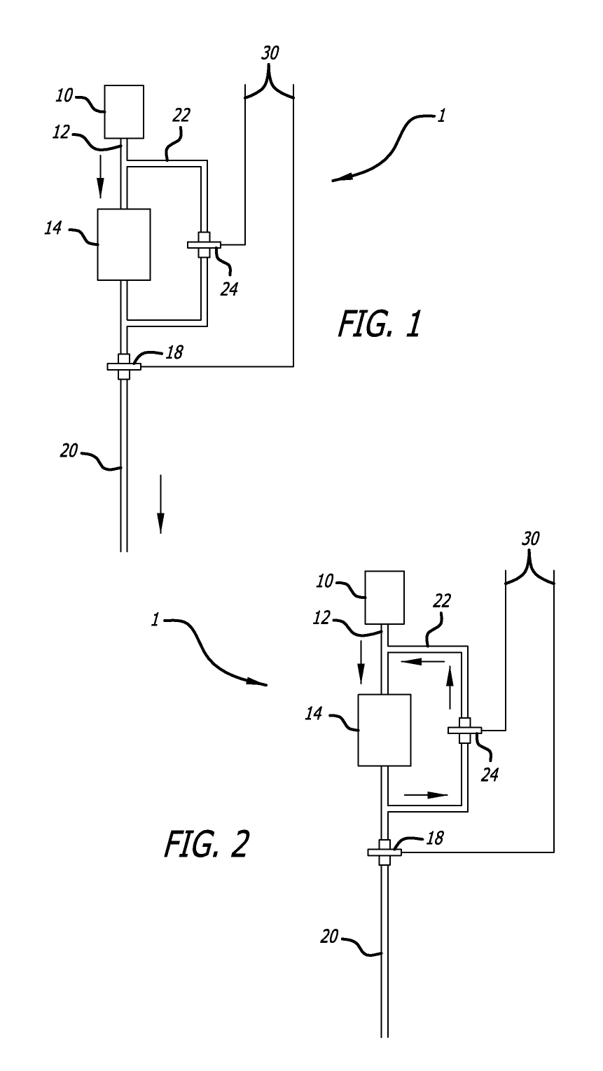

With reference to FIG. 1, the components and configuration of a fluid flow system 1 are shown for one embodiment of the present invention. A fluid bag 10 is connected to a pump input line 12 that goes to an inlet of an infusion pump 14. An outlet of the infusion pump 14 is connected to a patient line 20. Within the patient line 20 a valve 18, e.g. a pinch valve, is employed to regulate flow of fluid to the patient. The interface of the patient line 20 and a patient access point, such as an intravenous or other fluid flow path access, is not shown. The fluid system 1 further comprises a bypass line 22 having one end connected to the pump input line 12 and a second end connected to the patient line 20 between the infusion pump 14 and the valve 18. Within the bypass line 22 a valve 24, e.g. a pinch valve, is employed to regulate flow of fluid through the bypass line 22. The arrows in FIG. 1 show the fluid flow path when the fluid from the fluid bag 10 is intended to flow to the patient access point. When fluid is intended to flow to the patient, the patient line 20 valve 18 is in an open state, and the bypass line 22 valve 24 is in a closed state.

The arrows in FIG. 2 show the fluid flow path of the fluid flow system 1 when it is intended that fluid flow to the patient should stop. When fluid flow to the patient is intended to stop, the fluid flow through the patient line 20 is diverted to a bypass line 22 by closing the valve 18 and opening the bypass valve 24. The bypass line 22 returns fluid flow back to the pump input line 12 where the diverted fluid again enters the infusion pump 14. When the valve 18 is in a closed position, the pump input line 12; the infusion pump 14; and the bypass line 22 create a loop through which fluid is diverted from the patient line 20, without changing the flow setting of the infusion pump 14.

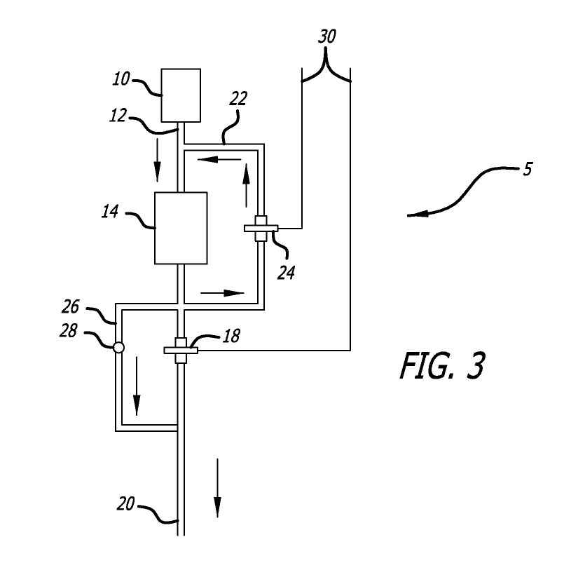

As previously mentioned, in certain situations, it may be necessary to maintain a low level of fluid flow through the patient line 20 to prevent the needle, catheter, or other patient access point (not shown) from clotting closed, i.e. a KVO fluid flow. FIG. 3 shows the components and configuration of a fluid flow system 5 for one embodiment of the present invention. The arrows in FIG. 3 show the fluid flow paths for system 5 when it is intended that fluid flow to the patient through patient line 20 be stopped and only a KVO fluid flow be maintained to the patient. In fluid flow system 5, a, KVO line 26 bypasses the valve 18 employed in the patient line 20. The KVO line 26 has a flow resistor 28 that reduces fluid flow to a lower, KVO fluid flow rate. Accordingly, when it is intended that fluid flow to the patient should be stopped, a portion of the fluid is circulated through the infusion pump 14 via bypass line 22, while a different portion of the fluid continues to flow to the patient at a KVO fluid flow rate through KVO line 26.

It is noted that the pinch valve 18 and the bypass pinch valve 24 are maintained in opposite states at substantially all times. Stated alternatively, when the fluid from the fluid bag 10 is intended to go to the patient the valve 18 is in an open state and the bypass valve 24 is in a closed state. On the other hand, when it is intended that fluid flow to the patient should be stopped, the valve 18 is in a closed state and the bypass valve 24 is in an open state.

In certain embodiments of the present invention, the open or closed states of the valve 18 and the bypass valve 24 are controlled by a signal provided by wires 30 that are connected at one end to valves 18 and 24 and at another end to a control instrument (not shown) that, in conjunction with a sensor (not shown), monitors one or more patient parameters. It will be understood by one of ordinary skill in the art that the wires 30 may be replaced by wireless forms of communication to achieve the same objectives. It will also be understood that sensor electronics may be incorporated into a patient monitor that is in turn in communication with the valve 18 and the bypass valve 24 directly or via a control instrument.

Certain embodiments of the present invention are utilized, for example, to control fluid flow to the brain through a ventricular catheter. In operation, when a first sensor detects that the requisite amount of fluid has been delivered to the brain, fluid flow through the ventricular catheter is stopped by closing the patient line valve and opening the bypass line valve. When a second sensor indicates that the injected fluid volume has been returned to the fluid source reservoir, the control unit determines that it is safe and/or desirable to allow another injection of fluid though the ventricular catheter. As the first and second sensors, the system may, for example employ infrared sensors operable to sense fluid levels and to convey a corresponding signal to the control unit. The control unit may, in turn, employ software that directs the flow state of the patient line and bypass valves. The control unit may additionally provide data output to the user regarding the flow status of the system.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

1. A system for delivering fluid to a patient comprising:
an infusion pump;
a fluid input flow path connecting a fluid reservoir to an infusion pump inlet;
a fluid output flow path having a output valve and connecting an infusion pump outlet to a patient access point; and
a first bypass fluid flow path having a first end in fluid communication with the fluid input flow path, a second end in fluid communication with the fluid output flow path, and a bypass valve.

2. The system for delivering fluid to a patient of claim 1 further comprising a second bypass fluid flow path having a first end in fluid communication with the fluid output flow path upstream of the output valve and a second end in fluid communication with the fluid output flow path downstream of the output valve.

3. The system for delivering fluid to a patient of claim 2 wherein the second bypass fluid flow path has a flow resistor.

4. The system for delivering fluid to a patient of claim 1 wherein at least one of the output valve and the bypass valve is a pinch valve.

5. The system for delivering fluid to a patient of claim 1 further comprising a control unit that controls the fluid flow state of at least one of the output valve and the bypass valve based upon a signal received from one or more sensors.

6. The system for delivering fluid to a patient of claim 5 wherein the sensor is a blood glucose monitor.

7. The system for delivering fluid to a patient of claim 1 wherein the patient access point is in fluid communication with a patient fluid flow path.

8. A method for intermittently delivering intravenous fluid to a patient comprising:
providing a fluid to an infusion pump inlet;
routing an output fluid flow from the infusion pump to a patient access point;
receiving a signal from a control instrument;
diverting the output fluid flow to the patient access point to the infusion pump inlet based on the signal from the control instrument.

9. The method of claim 8 wherein the step of providing a fluid to an infusion pump comprises routing an input fluid flow from a fluid reservoir to the infusion pump inlet.

10. The method of claim 8 wherein the step of routing an output fluid flow from the infusion pump to a patient access point comprises routing the output fluid flow through an output valve.

11. The method of claim 8 wherein the step of receiving a signal from a control instrument comprises receiving a signal at the output valve.

12. The method of claim 8 wherein the step of receiving a signal from a control instrument comprises receiving a signal emanating from a glucose sensor.

13. The method of claim 8 wherein the step of diverting the output fluid flow to the patient access point to an infusion pump inlet comprises routing the output fluid flow from an infusion pump outlet to the infusion pump inlet.

14. The method of claim 8 wherein the step of diverting the output fluid flow to the patient access point to an infusion pump inlet comprises circulating the fluid through the infusion pump.

15. The method of claim 8 wherein the step of diverting the output fluid flow to the patient access point to an infusion pump inlet comprises routing the output fluid flow through a bypass valve.

16. The method of claim 8 wherein the step of diverting the output fluid flow to the patient access point comprises diverting only a portion of the fluid flow to the patient access point.

17. The method of claim 16 wherein the step of diverting only a portion of the fluid flow to the patient access point comprises routing a remainder of the fluid flow to a patient access point through a fluid flow resistor.

18. A system for delivering fluid to a patient comprising:
a infusion pump;
a first fluid flow path having a first valve and a first end connected to an infusion pump outlet and a second end connected to a patient access point;
a second fluid flow path having a second valve and a first end connected to the infusion pump outlet and a second end connected to a infusion pump inlet; and
a sensor that senses a patient parameter and communicates a signal that affects a flow state of the first and second valves.

19. The system for delivering fluid to a patient of claim 18 further comprising a forth fluid flow path having a first end connected to the infusion pump outlet and a second end connected to the first fluid flow path downstream of the first valve.

20. The system for delivering fluid to a patient of claim 18 wherein the signal is communicated through a valve control unit.
